(19) United States
(12) Patent Application Publication
(10) Pub. No.: US 2016/0340311 A1
(43) Pub. Date: Nov. 24, 2016

(54) RECOVERY AND/OR REUSE OF PALLADIUM CATALYST AFTER A SUZUKI COUPLING

(71) Applicant: Dow AgroSciences LLC, Indianapolis, IN (US)

(72) Inventors: Sanjib Biswas, Midland, MI (US); Reetam Chakrabarti, Collegeville, PA (US); Lauren M. Huffman, Midland, MI (US); Ronald B. Leng, Midland, MI (US); Abraham D. Schuitman, Midland, MI (US); Karin Spiers, Midland, MI (US); Alan L. Stottlemyer, Midland, MI (US); Jeffrey B. Epp, Noblesville, IN (US)

(73) Assignee: Dow AgroSciences LLC, Indianapolis, IN (US)

(21) Appl. No.: 15/159,973

(22) Filed: May 20, 2016

Related U.S. Application Data
(60) Provisional application No. 62/165,502, filed on May 22, 2015.

(51) Int. Cl. C07D 213/803; B01J 31/24; B01J 31/22; C07D 405/04

(52) U.S. Cl. CPC C07D 213/803; C07D 405/04; B01J 31/2404; B01J 31/2295; B01J 2231/4227; B01J 2531/824

(57) ABSTRACT

Methods for the recovery and/or reuse of palladium catalyst after a Suzuki coupling reaction in which two molecules are coupled are described.

### CROSS REFERENCE TO RELATED APPLICATIONS

This application claims the benefit of U.S. Provisional Patent Application Ser. No. 62/165,502 filed May 22, 2015, which is expressly incorporated by reference herein.

### BACKGROUND

Suzuki coupling reactions are well known and the use of palladium catalysts in Suzuki coupling reactions is well characterized. However, palladium catalysts used in Suzuki couplings are generally not readily recoverable from reaction products. Thus, while the use of palladium as a catalyst in Suzuki couplings is well characterized and highly efficient, the cost of palladium catalysts is often a disproportionate portion of the raw material costs.

### SUMMARY

Methods for recycling palladium in a Suzuki coupling reaction are described. In these methods, a first Suzuki coupling of a compound of Formula (II)

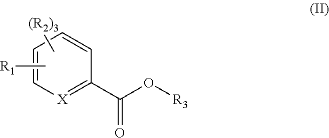

wherein
R₁ is halogen;
R₂ is H, halogen, —CN, —NO₂, formyl, C₁-C₆ alkyl, C₃-C₆ cycloalkyl, C₁-C₆ alkenyl, C₁-C₆ alkynyl, C₁-C₆ alkoxy, C₁-C₆ haloalkyl, C₁-C₆ haloalkenyl, C₁-C₆ haloalkynyl, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, aryloxy, heteroaryloxy, arylthio, heteroarylthio, NR₆R₇, or NHC(O)R₈;
R₃ is H, C₁-C₄ alkyl, or C₇-C₁₀ arylalkyl;
R₆, R₇ and R₈ are H or C₁-C₄ alkyl; and
X═CR₉ or N, wherein R₉ is H, halogen, NR₆R₇, or NHC(O)R₈,
   and a compound of Formula (III)

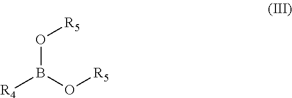

wherein
R₄ is a phenyl unsubstituted or substituted with 1-4 substituents independently selected from F, Cl, —CN, —NO₂, formyl, C₁-C₆ alkyl, C₃-C₆ cycloalkyl, C₁-C₆ alkenyl, C₁-C₆ alkynyl, C₁-C₆ alkoxy, C₁-C₆ haloalkyl, C₁-C₆ haloalkenyl, C₁-C₆ haloalkynyl, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, aryloxy, heteroaryloxy, arylthio, heteroarylthio, —NR₆R₇, or NHC(O)R₈ or a heteroaryl unsubstituted or substituted with from 1 to the maximum number of substituents independently selected from F, Cl, —CN, —NO₂, formyl, C₁-C₆ alkyl, C₃-C₆ cycloalkyl, C₁-C₆ alkenyl, C₁-C₆ alkynyl, C₁-C₆ alkoxy, C₁-C₆ haloalkyl, C₁-C₆ haloalkenyl, C₁-C₆ haloalkynyl, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, aryloxy, heteroaryloxy, arylthio, heteroarylthio, —NR₆R₇, or NHC(O)R₈;
R₅ is H, C₁-C₄ alkyl, or where the carbons on two R₅ are taken together to form a saturated ring as —O(C(R₁₀)₂)ₚO—, wherein p is 2 or 3; and
R₁₀ is H or C₁-C₄ alkyl,
   is performed. The Suzuki coupling reaction uses a palladium catalyst in the presence of a ligand and an amine base to form a first Suzuki coupling reaction product. The palladium catalyst is then substantially recovered from the first Suzuki coupling reaction product. The recovered palladium catalyst is then used in a second Suzuki coupling reaction.

Also described are methods for reclaiming palladium in a Suzuki coupling reaction. In these methods, a Suzuki coupling of a compound of Formula (II) and a compound of Formula (III) is performed. The Suzuki coupling reaction uses a palladium catalyst in the presence of a ligand and an amine base to form a Suzuki coupling reaction product. The palladium catalyst is then isolated from the Suzuki coupling reaction product into a palladium catalyst isolate. The palladium catalyst is then substantially reclaimed from the palladium catalyst isolate.

### BRIEF DESCRIPTION OF THE DRAWINGS

FIG. 1 shows a block diagram of the palladium recovery method of the present invention.

FIG. 2 shows a block diagram of one embodiment of the palladium recovery methods of the present invention.

FIG. 3 shows the palladium (Pd) concentration (in parts per million (ppm) on a dry weight basis) and the 4,5-dichloro-6-(4-chloro-2-fluoro-3-methoxyphenyl)picolinic acid (4,5-DCPA) concentration (in mole percent (mol %)) in the wash liquor based upon the acetonitrile (ACN)—water ratio (on a volume per volume (v/v) basis).

FIG. 4 shows the Pd concentration (ppm) on a dry weight basis and triethylamine (TEA) salt concentration (mol %) based upon the wash ratio.

FIG. 5 shows the Pd concentration (ppm) in dried 4,5-DCPA product.

FIG. 6 shows the TEA concentration (mol %) relative to dried 4,5-DCPA product.

### DETAILED DESCRIPTION

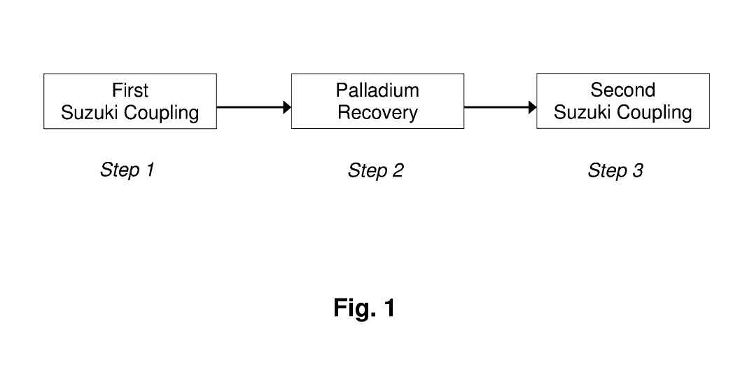

Methods for recycling palladium in a Suzuki coupling reaction are provided herein. In these methods, a first Suzuki coupling is performed in Step 1 and the palladium catalyst is then recovered from the reaction product of the first Suzuki coupling in Step 2. The recovered palladium catalyst is used in a second Suzuki coupling reaction in Step 3. FIG. 1 shows Steps 1 to 3. Palladium can be recovered on-site and reused immediately or palladium-containing material can be collected and the palladium reclaimed later (e.g., by a reclamation company). Typically, greater than 70% of the palladium catalyst is recovered and the recovered palladium is catalytically active.

### Suzuki Coupling

Suzuki coupling reactions are well known to those of skill in the art. As described herein, a molecule described by Formula (I) is the product of a Suzuki coupling:

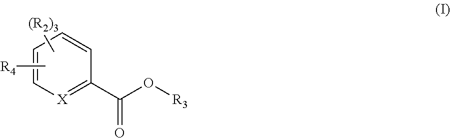

wherein

R₂ is H, halogen, —CN, —NO₂, formyl, C₁-C₆ alkyl, C₃-C₆ cycloalkyl, C₁-C₆ alkenyl, C₁-C₆ alkynyl, C₁-C₆ alkoxy, C₁-C₆ haloalkyl, C₁-C₆ haloalkenyl, C₁-C₆ haloalkynyl, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, aryloxy, heteroaryloxy, arylthio, heteroarylthio, NR₆R₇, or NHC(O)R₈;

R₃ is H, C₁-C₄ alkyl, or C₇-C₁₀ arylalkyl;

R₄ is a phenyl unsubstituted or substituted with 1-4 substituents independently selected from F, Cl, —CN, —NO₂, formyl, C₁-C₆ alkyl, C₃-C₆ cycloalkyl, C₁-C₆ alkenyl, C₁-C₆ alkynyl, C₁-C₆ alkoxy, C₁-C₆ haloalkyl, C₁-C₆ haloalkenyl, C₁-C₆ haloalkynyl, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, aryloxy, heteroaryloxy, arylthio, heteroarylthio, —NR₆R₇, or NHC(O)R₈ or a heteroaryl unsubstituted or substituted with from 1 to the maximum number of substituents independently selected from F, Cl, —CN, —NO₂, formyl, C₁-C₆ alkyl, C₃-C₆ cycloalkyl, C₁-C₆ alkenyl, C₁-C₆ alkynyl, C₁-C₆ alkoxy, C₁-C₆ haloalkyl, C₁-C₆ haloalkenyl, C₁-C₆ haloalkynyl, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, aryloxy, heteroaryloxy, arylthio, heteroarylthio, —NR₆R₇, or NHC(O)R₈;

R₆, R₇ and R₈ are H or C₁-C₄ alkyl; and

X═CR₉ or N, wherein R₉ is H, halogen, NR₆R₇, or NHC(O)R₈.

Unless specifically limited otherwise, the terms “alkyl”, “alkenyl” and “alkynyl”, as well as derivative terms such as “alkoxy”, “acyl”, “alkylthio”, “arylalkyl”, “heteroarylalkyl” and “alkylsulfonyl”, as used herein, include within their scope straight chain, branched chain and cyclic moieties. Thus, typical alkyl groups are methyl, ethyl, 1-methylethyl, propyl, 1,1-dimethylethyl, and cyclopropyl. Unless specifically stated otherwise, each may be unsubstituted or substituted with one or more substituents selected from but not limited to halogen, alkyl, alkenyl, alkynyl, hydroxy, alkoxy, alkylthio, C₁-C₆ acyl, formyl, cyano, aryloxy, or aryl, provided that the substituents are sterically compatible and the rules of chemical bonding and strain energy are satisfied. The term “haloalkyl” and “haloalkenyl” includes alkyl and alkenyl groups substituted with from one to the maximum possible number of halogen atoms, all combinations of halogens included. The terms “alkenyl” and “alkynyl” are intended to include one or more unsaturated bonds.

The term “aryl,” as used herein, refers to a phenyl, indanyl or naphthyl group. The term “heteroaryl,” as used herein, refers to a 5- or 6-membered aromatic ring containing one or more heteroatoms, viz., N, O or S; these heteroaromatic rings may be fused to other aromatic systems. Such heteroaromatic rings include, but are not limited to furanyl, thienyl, pyrrolyl, pyrazolyl, imidazolyl, triazolyl, isoxazolyl, oxazolyl, thiazolyl, isothiazolyl, pyridyl, pyridazyl, pyrimidyl, pyrazinyl and triazinyl ring structures. The aryl or heteroaryl substituents may be unsubstituted or substituted with one or more substituents selected from halogen, hydroxy, nitro, cyano, aryloxy, formyl, C₁-C₆ alkyl, C₂-C₆ alkenyl, C₂-C₆ alkynyl, C₁-C₆ alkoxy, halogenated C₁-C₆ alkyl, halogenated C₁-C₆ alkoxy, C₁-C₆ acyl, C₁-C₆ alkylthio, C₁-C₆ alkylsulfinyl, C₁-C₆ alkylsulfonyl, aryl, C₁-C₆OC(O)alkyl, C₁-C₆NHC(O)alkyl, C(O)OH, C₁-C₆C(O)Oalkyl, C(O)NH₂, C₁-C₆C(O)NHalkyl, or C₁-C₆C(O)N(alkyl)₂, provided that the substituents are sterically compatible and the rules of chemical bonding and strain energy are satisfied.

The term “arylalkyl,” as used herein, refers to a phenyl substituted alkyl group having a total of 7 to 11 carbon atoms, such as benzyl (—CH₂C₆H₅), 2-methylnaphthyl (—CH₂C₁₀H₇) and 1- or 2-phenethyl (—CH₂CH₂C₆H₅ or —CH(CH₃)C₆H₅). The phenyl group may itself be unsubstituted or substituted with one or more substituents independently selected from halogen, nitro, cyano, C₁-C₆ alkyl, C₁-C₆ alkoxy, halogenated C₁-C₆ alkyl, halogenated C₁-C₆ alkoxy, C₁-C₆ alkylthio, C(O)OC₁-C₆alkyl, or where two adjacent substituents are taken together as —O(CH₂)ₙO— wherein n=1 or 2, provided that the substituents are sterically compatible and the rules of chemical bonding and strain energy are satisfied.

Unless specifically limited otherwise, the term halogen includes fluorine, chlorine, bromine, and iodine.

In the Suzuki coupling reactions described herein, a compound of Formula (II) is reacted with a compound of Formula (III) to form a compound of Formula (I) as generally shown here:

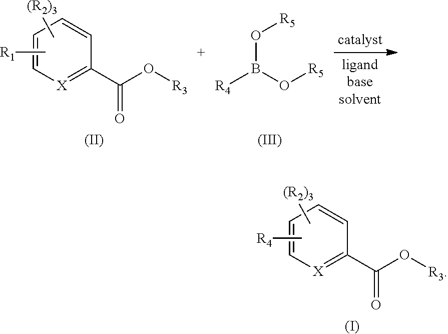

The compound of Formula (II) is

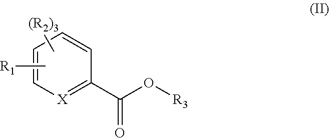

wherein

R₁ is halogen;

R₂ is H, halogen, —CN, —NO₂, formyl, C₁-C₆ alkyl, C₃-C₆ cycloalkyl, C₁-C₆ alkenyl, C₁-C₆ alkynyl, C₁-C₆ alkoxy, C₁-C₆ haloalkyl, C₁-C₆ haloalkenyl, C₁-C₆ haloalkynyl, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, aryloxy, heteroaryloxy, arylthio, heteroarylthio, NR₆R₇, or NHC(O)R₈;

R₃ is H, C₁-C₄ alkyl, or C₇-C₁₀ arylalkyl;

R₆, R₇ and R₈ are H or C₁-C₄ alkyl;

X═CR₉ or N, wherein R₉ is H, halogen, NR₆R₇, or NHC(O)R₈.

Optionally, when X is N, R₁ may be on the carbon ortho to the N. R₁ is noted to be halogen; however, the most common Suzuki coupling halogens are Cl, Br, and I and R₁ may be limited to Cl, Br, and/or I depending on the type of Suzuki coupling employed. Examples of compounds of Formula (II) include 5,6-dichloropicolinic acid; 4-bromobenzoic acid; methyl 5,6-dichloropicolinate; benzyl 5,6-dichloropicolinate; 3,4,5,6-tetrachloropicolinic acid; methyl 3,4,5,6-tetrachloropicolinate; benzyl 3,4,5,6-tetrachloropicolinate; 4-amino-3,5,6-trichloropicolinic acid; methyl 4-amino-3,5,6-trichloropicolinate; and benzyl 4-amino-3,5,6-trichloropicolinate.

The a compound of Formula (III) is

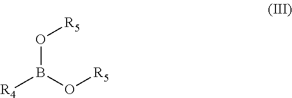

wherein

R₄ is a phenyl unsubstituted or substituted with 1-4 substituents independently selected from F, Cl, —CN, —NO₂, formyl, C₁-C₆ alkyl, C₃-C₆ cycloalkyl, C₁-C₆ alkenyl, C₁-C₆ alkynyl, C₁-C₆ alkoxy, C₁-C₆ haloalkyl, C₁-C₆ haloalkenyl, C₁-C₆ haloalkynyl, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, aryloxy, heteroaryloxy, arylthio, heteroarylthio, —NR₆R₇, or NHC(O)R₈ or a heteroaryl unsubstituted or substituted with from 1 to the maximum number of substituents independently selected from F, Cl, —CN, —NO₂, formyl, C₁-C₆ alkyl, C₃-C₆ cycloalkyl, C₁-C₆ alkenyl, C₁-C₆ alkynyl, C₁-C₆ alkoxy, C₁-C₆ haloalkyl, C₁-C₆ haloalkenyl, C₁-C₆ haloalkynyl, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, aryloxy, heteroaryloxy, arylthio, heteroarylthio, —NR₆R₇, or NHC(O)R₈;

R₅ is H, C₁-C₄ alkyl, or where the carbons on two R₅ are taken together to form a saturated ring as —O(C(R₁₀)₂)ₚO—, wherein p is 2 or 3; and

R₁₀ is H or C₁-C₄ alkyl.

Examples of compounds of Formula (III) include (2-fluoro-3-methoxyphenyl)boronic acid; phenylboronic acid; (4-chloro-2-fluoro-3-methoxyphenyl)boronic acid; furan-2-boronic acid; furan-2-boronic acid pinacol cyclic ester; and 4-chlorophenyl boronic acid.

Specific examples of R₄ as described herein are also described in International Application Nos. WO/2014/151005, WO/2014/151008, and WO/2014/151009 which are incorporated herein by reference.

A “palladium catalyst” as used herein is a palladium transition metal catalyst, such as palladium diacetate or bis(triphenylphosphine)palladium(II) dichloride. The palladium catalysts described herein can be prepared in situ from metal salts and ligands, such as palladium acetate and triphenylphosphine. Additional ligands useful with the methods described herein include bidentate ligands such as 1,3-bis(diphenylphosphino)propane (dppp), 1,1′-bis(diphenylphosphino)ferrocene (dppf), 1,1′-bis(di tert-butylphosphino)ferrocene (dtbpf), and 1,2-bis(diphenylphosphinomethyl)benzene and monodentate ligands such as (4-dimethyl-aminophenyl)phosphine (AmPhos), 2-dicyclohexylphosphino-2′,6′-dimethoxybiphenyl (SPhos), 2-dicyclohexylphosphino-2′,4′,6′-triisopropylbiphenyl (XPhos) and tri-o-tolylphosphine (TOTP). These in situ catalysts can be prepared by prior reaction of metal salt and ligand, followed by addition to the reaction mixture, or by separate addition of the metal salt and ligand directly to the reaction mixture.

Typically, Suzuki coupling reactions are carried out in the absence of oxygen using an inert gas, such as nitrogen or argon. Techniques used to exclude oxygen from coupling reaction mixtures, such as sparging with inert gas, are well known to those skilled in the art. Examples of such techniques are described in The Manipulation of Air-Sensitive Compounds, 2^{nd} ed.; Shriver, D. F., Drezdzon, M. A., Eds.; Wiley-Interscience, 1986. Sub-stoichiometric amounts of a catalyst are used, typically from about 0.0001 equivalents to 0.1 equivalents. Additional amounts of ligand may optionally be added to increase catalyst stability and activity. In addition, additives such as secondary or tertiary amine bases (such as triethylamine, diethylamine, pyridine, Hunig's base, diisopropylamine, and aromatic amines) and inorganic bases (such as Cs₂CO₃, Na₂SO₄, Na₂B₄O₇ and Na₂CO₃, K₂CO₃, KF, CsF, K₂HPO₄, K₃PO₄ and NaF) can be added to the coupling reaction. The coupling reaction generally requires from about 1 to about 5 equivalents of such additive, from 1 to 4.5 equivalents of such additive, from 1 to 4 equivalents of such additive, from 1 to 3.5 equivalents of such additive, from 1 to 3 equivalents of such additive, from 1 to 2.5 equivalents of such additive, from 1 to 2 equivalents of such additive, from 2 to 5 equivalents of such additive, from 2 to 4.5 equivalents of such additive, from 2 to 4 equivalents of such additive, from 2 to 3.5 equivalents of such additive, from 2 to 3 equivalents of such additive, from 3 to 5 equivalents of such additive, from 3 to 4.5 equivalents of such additive, or from 3 to 4 equivalents of such additive. Water may optionally be added to the coupling reaction to increase the solubility of the additives. The coupling reaction generally requires from 1 to about 3 equivalents of the compound of Formula (III), in some embodiments, from 1 to 1.5 equivalents. In some embodiments, sub-stoichiometric amounts of boronic acid may be used, e.g., greater than or equal to 0.85, greater than or equal to 0.9, greater than or equal to 0.91, greater than or equal to 0.92, greater than or equal to 0.93, greater than or equal to 0.94, greater than or equal to 0.95, greater than or equal to 0.96, greater than or equal to 0.97, greater than or equal to 0.98, or greater than or equal to 0.90 equivalents of the compound of Formula (III). The reaction is carried out in a solvent or mixture of solvents, such as acetone, acetonitrile, dimethyl sulfoxide (DMSO), dimethylformamide (DMF), dioxane, tetrahydrofuran (THF), methyl t-butyl ether (MTBE), xylenes, toluene, methylisobutyl ketone (MIBK), methanol, ethanol, isopropanol, butanol, or t-amyl alcohol (e.g., the reaction may be carried out in a mixture of acetonitrile and water). The temperature at which the reaction is conducted is not critical but usually is from about 25° C. to about 150° C. and, in some embodiments, from about 50° C. to about 125° C. A typical reaction generally requires from about 0.5 to about 24 hours. No particular order of addition of reactants is typically required. The reaction conditions can be controlled by controlled (e.g., continuous) addition of one or more reactants. In one embodiment, the compound of Formula (III) is added to the other reactants over several hours and the mixture is allowed to react for several more hours after the final addition of the compound of Formula (III).

### Palladium Recovery

After the Suzuki coupling reaction is completed, palladium is recovered in Step 2. One feature of the methods described herein, is that the palladium catalyst remains soluble over a very broad pH range, i.e., pH 0.1 to 14, so the palladium remains soluble and can be removed from the Suzuki coupling reaction product during the process of isolating the product. The pH over which the palladium can remain soluble can range from pH 0.1 to 13, pH 0.1 to 12, pH 0.1 to 11, pH 0.1 to 10, pH 0.5 to 14, pH 0.5 to 13, pH 0.5 to 12, pH 0.5 to 11, pH 0.5 to 10, pH 1 to 14, pH 1 to 13, pH 1 to 12, pH 1 to 11, pH 1 to 10, pH 2 to 14, pH 2 to 13, pH 2 to 11, pH 2 to 12, or pH 2 to 10.

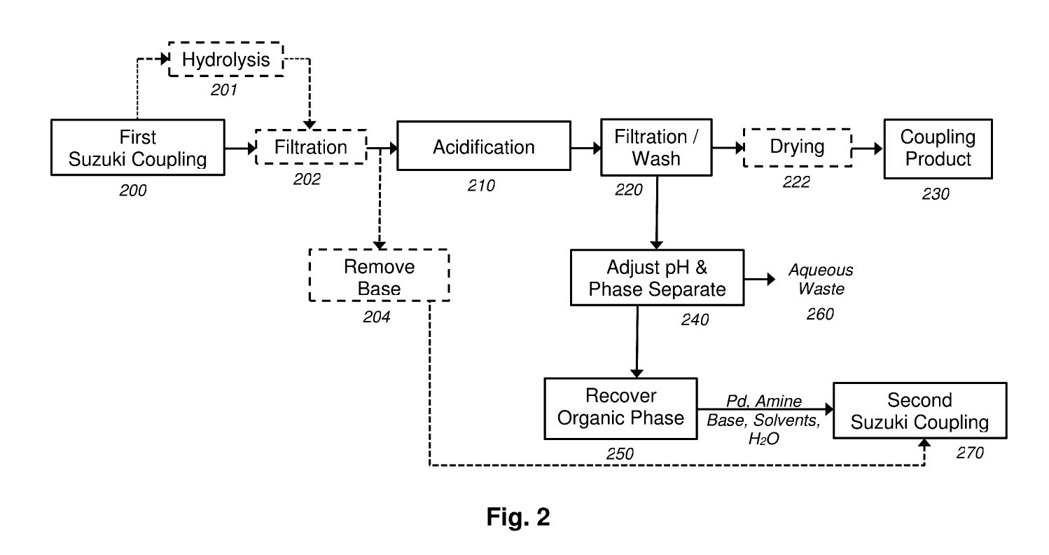

One method for recovery of the palladium catalyst from a Suzuki coupling reaction of a compound of Formula (II) and a compound of Formula (III) is shown in FIG. 2. In FIG. 2, a first Suzuki coupling 200 is performed as described above and the first Suzuki coupling product 230 is isolated from the reaction mixture. The first step in isolating the Suzuki coupling product and recovering the palladium catalyst is to acidify 210 the reaction mixture. The acid is used to neutralize the free base (e.g., triethylamine) and separate the Suzuki coupling product from complexes between the coupling product and base. Acids useful with the methods described herein will be apparent to those of skill in the art and include, but are not limited to, sulfuric acid, hydrochloric acid, and formic acid. The pH range achieved during the acidification step can range from pH 0.1 to pH 4 and can be calibrated to provide the most efficient separation of the Suzuki coupling product from the product—base complexes (if such complexes are present) without degrading the Suzuki coupling product 230. Once the Suzuki coupling product is separated from the base complexes, the coupling product will precipitate from solution. The temperature of the product mixture can be elevated during acidification 210 to help aid the separation of product—base complexes (e.g., 40-65° C.). The acidification 210 step is maintained until the Suzuki coupling reaction product is separated from the product—base complexes. Once the acidification reaction has separated the product—base complexes the Suzuki coupling product can precipitate out of solution. To aid precipitation, the temperature of the mixture can be lowered to reduce the solubility of the Suzuki coupling product 230. At this point in the palladium recovery effort, the palladium catalyst is distributed throughout the reaction mixture (mother liquor) and also mixed in with the precipitated Suzuki coupling product.

The next step 220 is to filter the reaction mixture to separate the precipitated Suzuki coupling product 230 from the mother liquor and wash the Suzuki coupling product 230 to remove any palladium catalyst. The separated mother liquor is placed in a palladium recovery vessel and the precipitated Suzuki coupling product 230 is washed with a mixture of a miscible aprotic solvent and water (e.g., an acetonitrile—water mixture can be used). The ratio of miscible aprotic solvent to water used for washing the precipitated Suzuki coupling product 230 can be balanced to minimize dissolution of the product while maximizing removal of the palladium. The ratio will depend on the solubility properties of the precipitated Suzuki coupling product 230 and the palladium catalyst. Examples of volume to volume ratios of miscible aprotic solvent to water include, but are not limited to, 95/5, 90/10, 85/15, 80/20, 75/25, 70/30, 65/35, 60/40, 55/45, 50/50, 45/55, 40/60, 35/65, 30/70, 25/75, 20/80, 15/85, 10/90, and 5/95. A further example of a useful miscible aprotic solvent to water mixture is a 50/50 volume to volume mixture of acetonitrile/water. The washings of the precipitated Suzuki coupling product are added to the palladium recovery vessel and the Suzuki coupling product can be dried. After washing and optionally drying 222, the Suzuki coupling product 230 is isolated from the reaction mixture and ready to be further purified or used in the manner intended.

Recovery of the palladium catalyst continues in the palladium recovery vessel by adjusting the pH to begin a phase-separation 240 of the combined mother liquor and washings. A base (aqueous or solid) is added to the mother liquor and washing mixture, which neutralizes any remaining amine base complexes and boric acid that were generated during the acidification step 210. Bases useful with the methods described herein will be apparent to those of skill in the art and include, but are not limited to, ammonium hydroxide, sodium hydroxide, and potassium hydroxide. Enough aqueous base is added to raise the pH such that two liquid phases are created, an aqueous phase 260 containing primarily water and inorganic salts and an organic-rich layer 250. The pH range at which such phase separation occurs is often in the pH 7-14 range, but can be a lower pH. In some embodiments, the pH can be greater than or equal to 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, or 13.0. The pH range at which such phase separation occurs can also be pH 1-7, pH 1-6, pH 1-5, pH 1-4, pH 1-3, pH 1-2, pH 2-7, pH 3-7, pH 4-7, pH 5-7, pH 2-6, pH 3-5, pH 6-14, 6-13, pH 6-12, pH 6-11, pH 6-10, pH 6-9, pH 6-8, pH 6-7, pH 7-14, 7-13, pH 7-12, pH 7-11, pH 8-10, pH 7-9, pH 7-8, pH 8-14, 8-13, pH 8-12, pH 8-11, pH 8-10, pH 8-9, pH 9-14, pH 9-13, pH 9-12, pH 9-11, pH 9-10, pH 10-14, pH 10-13, pH 10-12, or pH 10-11. It is possible that phase separation can occur without adjusting the pH by adding the base, however, palladium partitioning into the organic-rich layer tends to increase at higher pH levels. For example, if enough water is introduced into the palladium recovery vessel via the precipitated Suzuki coupling product 230 washings, phase separation could begin to occur, but as noted the palladium partitioning into the organic-rich layer may not be maximized and raising the pH by adding the base can be beneficial to palladium recovery. The temperature can be controlled, i.e., lowered to aid phase separation or raised to enable solute migration between phases, as needed (i.e., some water might partition into the organic-rich layer or organics into the aqueous layer). The aqueous layer 260 does not generally contain any useful reagents and is discarded, but could be further processed to recover solvent or reagents as desired. The organic-rich layer 250 contains the substantial majority of the palladium catalyst used in the Suzuki coupling reaction. The organic rich layer can contain greater than 60%, greater than 65%, greater than 70%, greater than 75%, greater than 80%, greater than 85%, greater than 86%, greater than 87%, greater than 88%, greater than 89%, greater than 90%, greater than 91%, greater than 92%, greater than 93%, greater than 94%, greater than 95%, greater than 96%, greater than 97%, greater than 98%, greater than 99% of the original amount of palladium catalyst used in the Suzuki coupling reaction. As used herein, the term “substantially recovering” means recovering the majority of the palladium catalyst used in the Suzuki coupling reaction, i.e., recovering greater than 60%, greater than 75%, greater than 70%, greater than 75%, greater than 80%, greater than 85%, greater than 86%, greater than 87%, greater than 88%, greater than 89%, greater than 90%, greater than 91%, greater than 92%, greater than 93%, greater than 94%, greater than 95%, greater than 96%, greater than 97%, greater than 98%, greater than 99% of the original amount of palladium catalyst used in the Suzuki coupling reaction.

In addition to palladium catalyst, the organic-rich layer contains solvents and reactants used in the Suzuki coupling reaction and as such could be directly added to a second Suzuki coupling reaction. Alternatively, the palladium could be recovered and reconstituted into a useful catalyst. The organic-rich layer can be used directly in a Suzuki coupling reaction with similar reagents or sent to a palladium reclamation service provider to isolate the palladium. When the organic rich-layer is directly added to a second Suzuki coupling reaction, the palladium catalyst is still active, but the catalytic rates may be decreased (other ligands may also be present in the organic-rich layer and would be available to react). Catalytic rates of recycled palladium catalyst can be greater than 40%, greater than 45%, greater than 50%, greater than 55%, greater than 60%, greater than 65%, greater than 70%, greater than 75%, greater than 80%, greater than 85%, greater than 90%, or greater than 95%. The methods discussed herein have been framed with respect to a first Suzuki coupling reaction and a second Suzuki coupling reaction, however, it is intended that the palladium recovery methods can equally be applied to the palladium used in the second Suzuki coupling reaction, which could be recycled into a third Suzuki coupling reaction. Palladium can be recovered using the methods described herein and used indefinitely in subsequent reactions. In fact, with high palladium recovery levels, many Suzuki coupling reactions could be performed using the same palladium that is recycled using the methods described herein after each reaction.

Additional options are available during palladium catalyst recovery and Suzuki coupling product isolation. One option, when R₃ is not H, is to perform a hydrolysis step 201 prior to the acidification 210. Another option is to filter 202 the reaction mixture prior to the acidification step 210 in order to remove any solid byproducts that formed during the Suzuki coupling reaction (such filtration methods will be apparent to those of skill in the art). The hydrolysis step 201 can be done before the filtration step 202. A further option available during palladium catalyst recovery and Suzuki coupling product isolation is to remove 204 non-complexed base from the reaction mixture prior to the acidification 210 step in order to simplify the workup of the reaction mixture (i.e., acidification 210 step will not require as much acid if lower amounts of base are present to neutralize). Distillation is one method to remove amine base 204 prior to the acidification 210 step, but other methods will be apparent to those of skill in the art. A further option is to process the organic-rich layer after recovery 250 to separate Suzuki coupling reaction components such as the amine base (e.g., triethylamine) and solvent (e.g., acetonitrile) to generate a more concentrated palladium-containing phase. Distillation of the organic-rich phase is one option in which amine bases and solvents can be separated while leaving a further concentrated palladium-rich phase. The recovered amine bases and solvents can optionally be reused in further Suzuki coupling reactions or in other steps (e.g., recovered acetonitrile could be reused in the post-acidification wash step). The concentrated palladium-rich phase could be internally processed, sent to a palladium reclamation service provider, or recycled directly to a second Suzuki coupling reaction. Additional options for recovering palladium would include adding an organic solid substrate (e.g., carbon black, diatomaceous earth, or other material that can be removed during palladium reclamation) to the palladium-rich phase or organic-rich phase to adsorb the palladium onto the surface of the organic solid substrate material and removing the solid substrate from the remaining palladium rich phase or organic-rich phase, e.g., by filtration, then reclaiming the palladium from the solid substrate. A further option is to add water to the organic-rich phase and isolate the palladium as a solid.

The recovery of palladium from Suzuki coupling reactions of the following general scheme is described herein:

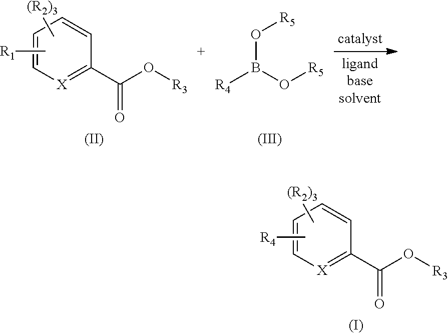

Table 1 contains examples of possible compounds of Formulae (II) and (III), catalysts, ligands, bases and solvents that can be combined in the above reaction scheme. Some of the combinations suggested in Table 1 are used in the experimental procedures described below.

TABLE 1
Examples of Suzuki Coupling Optional Components

| Compound of | Compound of | | | |
|---|---|---|---|---|
| Formula (II) | Formula (III) | Catalyst and Ligand | Base | Solvents |
| 5,6- | (2-fluoro-3- | Pd(OAc)₂ and | diethylamine | ACN |
| dichloropicolinic | methoxyphenyl)- | triphenylphosphine | | |
| acid | boronic acid | | | |
| 4-bromobenzoic | phenylboronic | Pd(OAc)₂ and dppf | triethylamine | THF |
| acid | acid | | | |
| methyl 5,6- | furan-2-boronic | Pd(OAc)₂ and dppp | pyridine | Acetone |
| dichloropicolinate | acid pinacol | | | |
| | cyclic ester | | | |
| benzyl 5,6- | 4-chlorophenyl | Pd(OAc)₂ and 1,2- | cesium | DMSO |
| dichloropicolinate | boronic acid | bis(diphenylphosphino- | carbonate | |
| | | methyl)benzene | | |
| 3,4,5,6-tetrachloro- | | Pd(OAc)₂ and XPhos | Hunig's Base | Dioxane |
| picolinic acid | | | | |
| methyl 3,4,5,6- | | Pd(OAc)₂ and AmPhos | | DMF |
| tetrachloro- | | | | |
| picolinate | | | | |
| benzyl 3,4,5,6- | | Pd(OAc)₂ and SPhos | | MIBK |
| tetrachloro- | | | | |
| picolinate | | | | |
| 4-amino-3,5,6- | | (dtbpf)PdCl₂ | | Methanol |
| trichloropicolinic | | | | |
| acid | | | | |
| methyl 4-amino- | | (AmPhos)₂PdCl₂ | | |
| 3,5,6- | | | | |
| trichloropicolinate | | | | |
| benzyl 4-amino- | | | | |
| 3,5,6- | | | | |
| trichloropicolinate | | | | |
| 5,6- | furan-2-boronic | Pd(OAc)₂ and | triethylamine | ACN- |
| dichloropicolinic | acid | triphenylphosphine | | water |
| acid | | | | |
| 5,6- | (4-chloro-2- | Pd(OAc)₂ and dppf | triethylamine | ACN- |
| dichloropicolinic | fluoro-3- | | | water |
| acid | methoxyphenyl)- | | | |
| | boronic acid | | | |
| 4,5,6- | (4-chloro-2- | Pd(OAc)₂ and | triethylamine | ACN- |
| trichloropicolinic | fluoro-3- | triphenylphosphine | | water |
| acid | methoxyphenyl)- | | | |
| | boronic acid | | | |

The described compositions and methods and following examples are for illustrative purposes and are not intended to limit the scope of the claims. Other modifications, uses, or combinations with respect to the compositions and methods described herein will be apparent to a person of ordinary skill in the art without departing from the spirit and scope of the claimed subject matter.

### EXAMPLES

### Example 1

### Efficacy of Acetonitrile-Water Washing

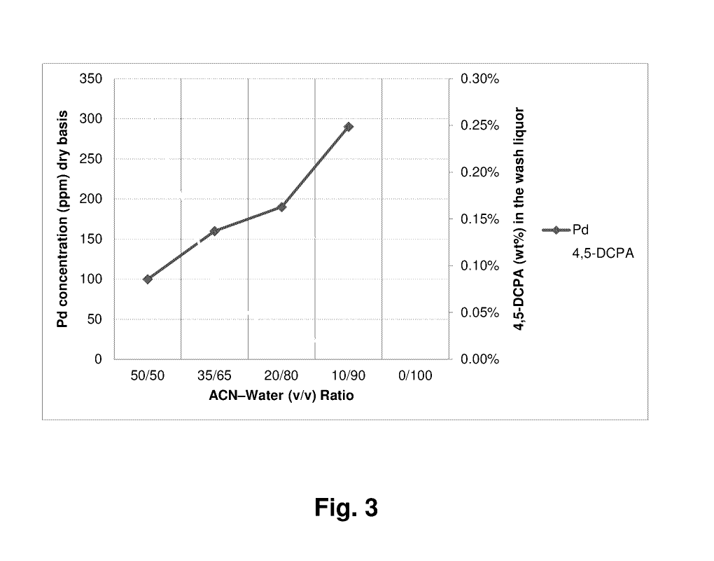

Acidified 4,5-Dichloro-6-(4-chloro-2-fluoro-3-methoxyphenyl)picolinic acid (4,5-DCPA) product slurry (pH=0.5, temperature ˜10° C., held at temperature for ˜6 hours (h)) was split into multiple batches and each batch was washed in a centrifuge with 3 bed volumes of different concentrations of acetonitrile (ACN)—water mixtures. The palladium (Pd) concentration in dried 4,5-DCPA product and the concentration of 4,5-DCPA in the mother-liquors and wash were recorded (FIG. 3). Higher ACN concentrations result in lower Pd concentration in dried 4,5-DCPA product. However, this also lowers yield of isolated 4,5-DCPA product due to higher solubility losses in mother liquors and wash. Therefore, an optimum concentration can be used depending on desired need.

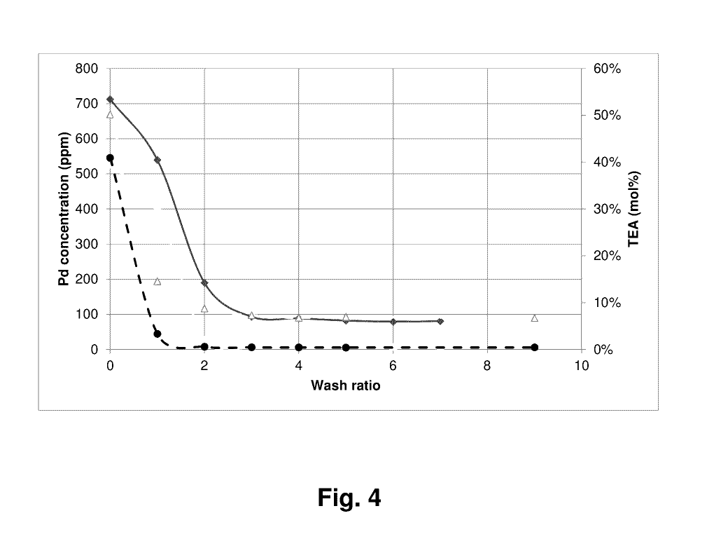

Two batches of 4,5-DCPA product (pH=0.5, temperature 10-15° C., held at low temperature for 30 minutes (min) and ˜6 h) were filtered and washed with multiple bed volumes of 50/50 volume per volume (v/v) ACN—water (as shown in FIG. 4). In a single bed-volume, the ACN content was ˜0.58 mass ratio to 4,5,6-trichloropicolinic acid (4,5,6-TCPA), while the water was ˜0.75 mass ratio to 4,5,6-TCPA. Pd and TEA concentrations in dried 4,5-DCPA product were measured after each wash (FIG. 4). Each wash ratio corresponds to one-bed volume of 50/50 (v/v) ACN—water. Based on the data collected, the washing procedure was optimized and the number of washes was reduced to 3 bed volumes of ACN—water wash followed by water washes as necessary to increase the pH of the wetcake. Pd concentrations are shown as diamonds and triangles in FIG. 4. TEA concentrations are shown as squares and circles in FIG. 4. Batch 1 (filtration after holding the slurry for ˜30 min at 15° C.) is shown as a solid line in FIG. 4 and Batch 2 (filtration after holding the slurry ˜6 h at 10-15° C.) is shown as a dashed line in FIG. 4.

### Example 2

### Efficacy of Acetonitrile-Water Washing

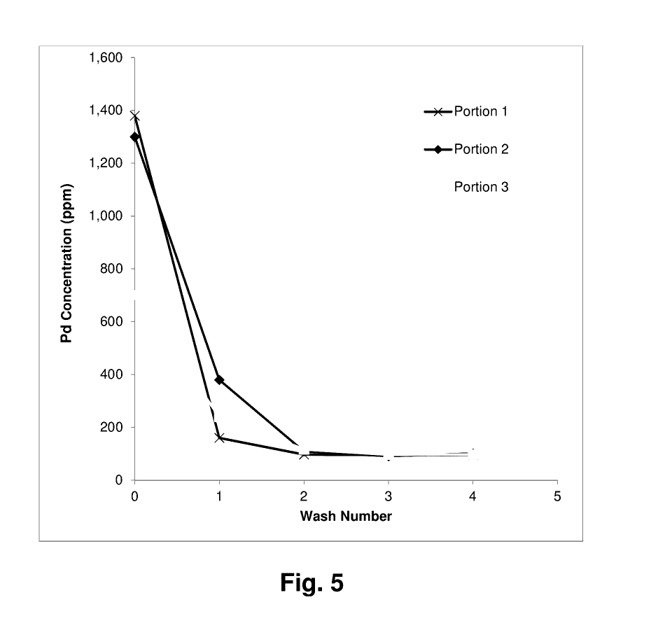

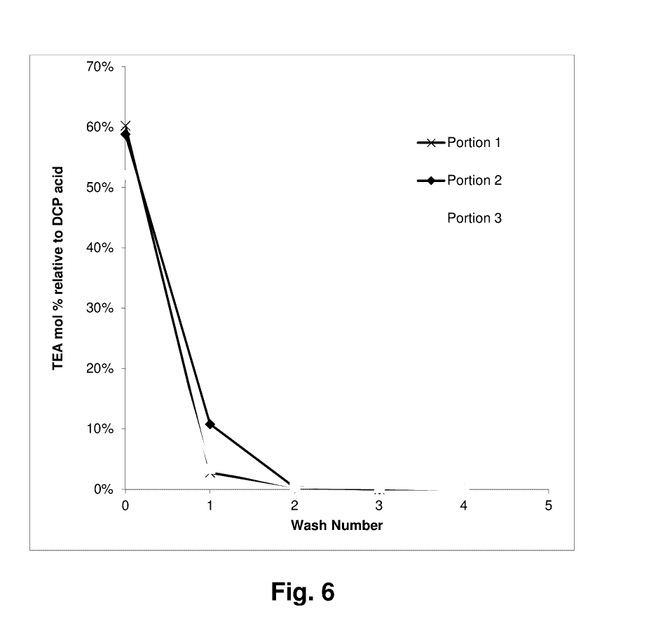

Acidified 4,5-Dichloro-6-(4-chloro-2-fluoro-3-methoxyphenyl)picolinic acid (4,5-DCPA) product slurry (pH=2.7, temperature 5° C., held at temperature for ˜6 h) was filtered in three portions (lower acid concentration and higher pH than Example 1) (see FIGS. 5 and 6). One precipitation mixture portion is one-third of a batch. Each portion was washed with three bed volumes of 50/50 (v/v) ACN—water. In a single bed-volume, the ACN content was ˜0.58 mass ratio to 4,5,6-TCPA, while the water was ˜0.75 mass ratio to 4,5,6-TCPA). The total ACN content was ˜1.75 mass ratio to 4,5,6-TCPA, while the water was ˜2.24 mass ratio to 4,5,6-TCPA. Each portion was then washed with a single bed volume of water (corresponding to 1.33 mass ratio to 4,5,6-TCPA). The variation in palladium (Pd) and triethylamine (TEA) concentrations in dried product with each wash was recorded (FIGS. 5 and 6) and was similar to Example 1. Most of the Pd and TEA is removed from the cake at the end of the second bed volume ACN—water wash resulting in ˜100 ppm Pd and <0.1 mol % TEA in the product (FIGS. 5 and 6). Most of the Pd from the Suzuki coupling step is therefore in the mother liquors and wash stream. If all the Pd were in the dried product, the concentration of Pd would be ˜5000 ppm. After the second and third washes, the 4,5-DCPA dried product contained <5% of the total Pd loaded into the reactor.

### Example 3

### Addition of Base to pH 12 with Concentration to Homogeneous Solution

A combined mother liquor and wash stream (430.85 g, 360 ppm Pd) from the isolation of 4,5-DCPA was neutralized and then made basic (pH 12) with sodium hydroxide (NaOH, 50 weight percent (wt %) solution in water; 43.23 g). The mixture separated into two phases. The top organic-rich phase (312.11 g, 480 ppm Pd) was retained, and the bottom, aqueous phase (157.07 g, <1 ppm Pd) was discarded. The top organic-rich phase was then distilled on a rotary evaporator until solids developed. The collected solvents were added back to the mixture until a homogeneous solution was obtained. The resulting mixture was 1260 ppm Pd, resulting in 99% recovery.

### Example 4

### Addition of Base to pH 12 with Concentration to Solid

A combined mother liquor and wash stream (217.8 g, 230 ppm Pd) from the isolation of 4,5-DCPA was neutralized and then made basic (pH 12) with NaOH (21.33 g) at room temperature. The mixture was kept in an oven at 55° C. for about 3 h. The mixture separated into two phases with a small interfacial layer at the interface. The mass of the aqueous phase was 79.05 g. The interfacial layer was collected separately. Solids were observed at the bottom of the organic-rich layer after cooling to room temperature. The solids were filtered (2.3 g), and the organic-rich phase (157.0 g) was transferred to a rotary evaporator and distilled to about 26.1 g remaining. During the concentration process, solids started to form. Water (40.0 g) was added to the concentrated organic-rich phase, and the solids were collected via filtration. The solids (3.53 g) contained 1.32 wt % Pd, resulting in 92.8% recovery from the parent mother liquors stream.

### Example 5

### Neutralization to pH 7

A combined mother liquor and wash stream (771.6 g, 400 ppm Pd) from the isolation of 4,5-DCPA was neutralized (pH 8) with 61.3 g of 28 wt % aqueous NH₄OH. A cloudy solution was obtained. Upon allowing the solution to settle at room temperature, the mixture separated into two phases. Both the top organic-rich layer and the bottom aqueous phase were analyzed. The top organic-rich layer (367.11 g, 650 ppm Pd, yellow in color) was concentrated, and the bottom, clear aqueous layer was discarded (463.02 g, 50 ppm Pd). The top organic-rich layer was concentrated to 41% of its initial mass, 151.9 g and 1510 ppm Pd, resulting in a 91% recovery from the top organic-rich layer to the concentrated layer, or a 74% overall recovery of palladium.

### Example 6

### Neutralization to pH 7

A combined mother liquor and wash stream (100 mL) from the isolation of 4,5-DCPA was neutralized (pH 7) with 50 wt % aqueous NaOH. A clear solution was obtained. Upon cooling the solution in an ice bath, the mixture separated into two phases. Both the top and the bottom layers were analyzed

The results are given below. All of the values are in wt % unless otherwise noted.

The organic-rich layer contained 67-68% ACN; 1.5% TEA; 1.4% 2-chloro-5-fluoroanisole; 0.15% 4,5,6-TCPA; 0.6% 4,5-DCPA; and ˜1000-1100 ppm Pd, corresponding to approximately 90% palladium recovery.

The aqueous layer contained 20-21% ACN; 3% TEA; 0% 2-chloro-5-fluoroanisole; 0.05% 4,5,6-TCPA; 0.06% 4,5-DCPA; and ˜10 ppm Pd.

### Example 7

### Catalyst Recycling with No Additional Ligand

A 250 mL-round bottom flask equipped with overhead stirring, nitrogen sparge, and temperature control was charged with 4,5,6-TCPA (7.99 g, 0.033 mol). The organic-rich layer from a neutralized mother liquor solution (1.5 mol % Pd, 98 g of a 1100 ppm Pd solution) was added to the flask. A solution of ACN (94 mL), water (36 mL), and TEA (14.5 mL) was prepared and added to the 250 mL-round bottom flask. The mixture was purged with nitrogen for 30 minutes (min) 4-Chloro-2-fluoro-3-methoxyphenyl)boronic acid (7.33 g, 0.036 mol) was added, and the mixture was sparged with nitrogen for 30 min, then padded with nitrogen and heated for 18 hours (h) at 65° C. The reaction progress was monitored by liquid chromatography (LC). 4,5-DCPA was produced in 57% in-pot yield. The remaining balance of material was 4,5,6-TCPA.

### Example 8

### Catalyst Recycling with Extra Ligand

A 250 mL-round bottom flask equipped with overhead stirring, nitrogen sparge, and temperature control was charged with 4,5,6-TCPA (10.03 g, 0.041 mol). The organic-rich layer from a neutralized mother liquor solution (1.5 mol % Pd, 120 g of a 1100 ppm Pd solution) was added to the flask. A solution of ACN (92 mL), water (44 mL) and TEA (15.9 mL) was prepared then added to the 250 mL-round bottom flask. The mixture was purged with nitrogen for 30 min. Triphenylphosphine (0.32 g) was added to make up for the balance of ligand presumed lost during the workup. 4-Chloro-2-fluoro-3-methoxyphenyl) boronic acid (9.13 g, 0.045 mol) was added, and the mixture was sparged with nitrogen for 30 min, then padded with nitrogen and heated to 65° C. for 18 h. The reaction progress was monitored by LC. The 4,5-DCPA was produced in 16% in-pot yield. The remaining material was unconverted 4,5,6-TCPA.

### Example 9

### Catalyst Recycling of Solids Obtained from Mother Liquors with No Additional Ligand and Constant Addition of Boronic Acid

A combined mother liquor wash stream generated as in Example 1 (730 g) was neutralized and then made basic (pH 8) with 29% aqueous ammonium hydroxide (NH₄OH; 69.43 g). The mixture separated into two layers; the top organic-rich layer was kept and the bottom, colorless layer was discarded. The top organic-rich layer was concentrated until yellow solids formed. The solids were isolated by filtration and washed with water. The solids were found to contain 1.97 wt % Pd. Other components of the solids were found to be 35 wt % 4,5-DCPA, 9 wt % of the isomer of 4,5,-DCPA, 6 wt % 4,5,6-TCPA, 2 wt % 5-chloro-4,6-bis(4-chloro-2-fluoro-3-methoxyphenyl)picolinic acid, and 3 area % 4,4′-dichloro-2,2′-difluoro-3,3′-dimethoxy-1,1′-biphenyl.

A 250 mL-round bottom flask equipped with overhead stirring, nitrogen sparge, and temperature control was charged with 4,5,6-TCPA (10.21 g, 0.041 mol). A solution of ACN (94 mL), water (36 mL) and TEA (14.5 mL) was prepared and then a portion of the solution (105 mL) was added to the 250 mL-round bottom flask containing the 4,5,6-TCPA. The solids dissolved, and the mixture was purged with nitrogen for 30 min. The above reclaimed palladium solids (3.05 g, corresponding to 1.4 mol % Pd loading) were added to the sparged solution in the 250 mL-round bottom flask, and the mixture was sparged for an additional 5 min Separately, a solution of (4-chloro-2-fluoro-3-methoxyphenyl)boronic acid (9.12 g, 0.045 mol) was prepared in the remaining (40 mL) ACN/water/TEA solution and was sparged with nitrogen for 30 min. The boronic acid solution was then loaded into a syringe pump for constant addition over 6 h. The reaction mixture was padded with nitrogen and heated to 65° C. for 18 h. The reaction progress was monitored by LC. The 4,5-DCPA was produced in 74% in-pot yield, with 4% of the isomer of 4,5,-DCPA, 6% 5-chloro-4,6-bis(4-chloro-2-fluoro-3-methoxyphenyl)picolinic acid. The remaining material was 16% unconverted 4,5,6-TCPA.

### Example 10

### Catalyst Recovery after Coupling of 5,6-Dichloropicolinic Acid and Furan-2-Boronic Acid

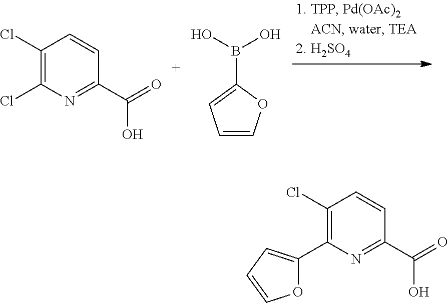 [SMILES: O=C(O)c1ccc(Cl)c(Cl)n1 ; OB(O)c1ccco1 ; O=C(O)c1ccc(Cl)c(-c2ccco2)n1]

To a 100 mL round bottom flask equipped with a magnetic stirrer, reflux condenser and a nitrogen inlet were added 5,6-dichloropicolinic acid (5.00 g, 23.1 mmol), TEA (8.2 g, 81.0 mmol), ACN (39.5 g) and water (15.1 g). The solution was sparged for 30 min with nitrogen (1 mL/min) After sparging, triphenylphosphine (TPP; 0.18 g, 0.686 mmol) and palladium(II) acetate (0.078 g, 0.347 mmol) were added to the solution. Furan-2-boronic acid (3.3 g, 28.9 mmol) was added in one portion, and heating was initiated. The reaction mixture was heated to 55° C., and was sampled and analyzed by liquid chromatography. No boronic acid was remaining after two hours, and heating was stopped. The reaction mixture was allowed to cool overnight and then was heated to 45° C. Once at temperature, 50% sulfuric acid (7.1 g) was added. No precipitation was observed, so the mixture was cooled. After 30 min at <5° C., no solids were observed and water (25.7 g) was added. A precipitate formed which was allowed to cool for 1 h and isolated by filtration. The flask was rinsed with cold mother liquor to isolate all of the product. The wetcake was then rinsed with cold ACN—water solution (8.75 g and 11.25 g, respectively). The palladium content was analyzed in the wetcake, wash and mother liquors, with 81% of the palladium in the mother liquor and wash, and 19% in the wet cake. 99% of the total palladium added was recovered.

### Example 11

### Catalyst Recovery after Coupling of 5,6-Dichloropicolinic Acid and (4-Chloro-2-fluoro-3-methoxyphenyl)boronic Acid

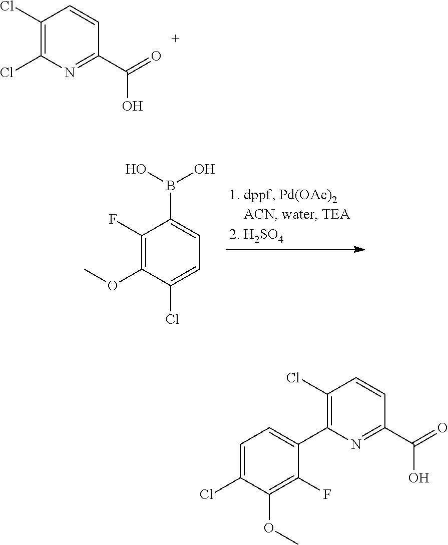 [SMILES: O=C(O)c1ccc(Cl)c(Cl)n1 ; COc1c(Cl)ccc(B(O)O)c1F ; COc1c(Cl)ccc(-c2nc(C(=O)O)ccc2Cl)c1F]

To a 100 mL round bottom flask equipped with a magnetic stirrer, reflux condenser and a nitrogen inlet were added 5,6-dichloropicolinic acid (5.00 g, 23.1 mmol), TEA (8.3 g, 81.0 mmol), ACN (39.9 g) and water (15.3 g). The solution was sparged for 30 min with nitrogen (1 mL/min) After sparging, 1,1′-bis(diphenylphosphino)ferrocene (dppf; 0.19 g, 0.343 mmol) and palladium(II) acetate (0.08 g, 0.356 mmol) were added to the solution. (4-Chloro-2-fluoro-3-methoxyphenyl)boronic acid 5.4 g, 26.9 mmol) was added in one portion, and heating was initiated. The reaction mixture was heated to 55° C., and was sampled and analyzed periodically by liquid chromatography. No boronic acid was remaining after 22 hours, and heating was stopped. The reaction mixture was allowed to cool to 45° C. Once at temperature, 50% sulfuric acid (7.2 g) was added. No precipitation was observed, so the mixture was cooled. A precipitate formed, which was isolated by filtration. The flask was rinsed with cold mother liquor to isolate all of the product. The wetcake was then rinsed with cold ACN—water solution (8.75 g and 11.25 g, respectively). The palladium content was analyzed in the wetcake, wash and mother liquors, with 96% of the palladium in the mother liquor and wash, and 4% in the wet cake. 98% of the total palladium added was recovered.

The present invention is not limited in scope by the embodiments disclosed herein which are intended as illustrations of a few aspects of the invention and any embodiments which are functionally equivalent are within the scope of this invention. Various modifications of the methods in addition to those shown and described herein will become apparent to those skilled in the art and are intended to fall within the scope of the appended claims. Further, while only certain representative combinations of the method steps disclosed herein are specifically discussed in the embodiments above, other combinations of the composition components and method steps will become apparent to those skilled in the art and also are intended to fall within the scope of the appended claims. Thus a combination of method steps may be explicitly mentioned herein; however, other combinations of method steps are included, even though not explicitly stated. The term “comprising” and variations thereof as used herein is used synonymously with the term “including” and variations thereof and are open, non-limiting terms.

What is claimed is:

1. A method for recycling palladium in a Suzuki coupling reaction comprising:
A) performing a first Suzuki coupling of a compound of Formula (II) 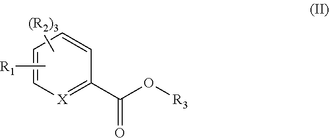
wherein
R₁ is halogen;
R₂ is H, halogen, —CN, —NO₂, formyl, C₁-C₆ alkyl, C₃-C₆ cycloalkyl, C₁-C₆ alkenyl, C₁-C₆ alkynyl, C₁-C₆ alkoxy, C₁-C₆ haloalkyl, C₁-C₆ haloalkenyl, C₁-C₆ haloalkynyl, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, aryloxy, heteroaryloxy, arylthio, heteroarylthio, NR₆R₇, or NHC(O)R₈;
R₃ is H, C₁-C₄ alkyl, or C₇-C₁₀ arylalkyl;
R₆, R₇ and R₈ are H or C₁-C₄ alkyl; and
X═CR₉ or N, wherein R₉ is H, halogen, NR₆R₇, or NHC(O)R₈,
and a compound of Formula (III) 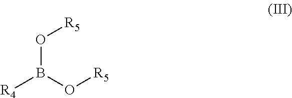
wherein
R₄ is a phenyl unsubstituted or substituted with 1-4 substituents independently selected from F, Cl, —CN, —NO₂, formyl, C₁-C₆ alkyl, C₃-C₆ cycloalkyl, C₁-C₆ alkenyl, C₁-C₆ alkynyl, C₁-C₆ alkoxy, C₁-C₆ haloalkyl, C₁-C₆ haloalkenyl, C₁-C₆ haloalkynyl, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, aryloxy, heteroaryloxy, arylthio, heteroarylthio, —NR₆R₇, or NHC(O)R₈ or a heteroaryl unsubstituted or substituted with from 1 to the maximum number of substituents independently selected from F, Cl, —CN, —NO₂, formyl, C₁-C₆ alkyl, C₃-C₆ cycloalkyl, C₁-C₆ alkenyl, C₁-C₆ alkynyl, C₁-C₆ alkoxy, C₁-C₆ haloalkyl, C₁-C₆ haloalkenyl, C₁-C₆ haloalkynyl, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, aryloxy, heteroaryloxy, arylthio, heteroarylthio, —NR₆R₇, or NHC(O)R₈;
R₅ is H, C₁-C₄ alkyl, or where the carbons on two R₅ are taken together to form a saturated ring as —O(C(R₁₀)₂)ₚO—, wherein p is 2 or 3; and
R₁₀ is H or C₁-C₄ alkyl,
using a palladium catalyst in the presence of a ligand and a base to form a first Suzuki coupling reaction product;
B) substantially recovering the palladium catalyst from the first Suzuki coupling reaction product; and
C) performing a second Suzuki coupling of a compound of Formula (II) and a compound of Formula (III) using the recovered palladium catalyst.

2. The method of claim 1, wherein greater than 70% of the palladium catalyst used in the first Suzuki coupling reaction is recovered.

3. The method of claim 1, wherein the palladium catalyst is formed from palladium acetate or palladium chloride.

4. The method of claim 1, wherein the substituted boronic acid is (4-chloro-2-fluoro-3-methoxyphenyl)boronic acid.

5. The method of claim 1, wherein R₃ is H.

6. The method of claim 1, wherein the compound of Formula (II) is chlorinated.

7. The method of claim 1, wherein the compound of Formula (II) is 4,5,6-trichloropicolinic acid or 5,6-dichloropicolinic acid.

8. The method of claim 1, wherein the ligand is triphenylphosphine or dppf.

9. The method of claim 1, wherein the Suzuki coupling reaction is done in a solvent system comprising a miscible polar aprotic solvent and water.

10. The method of claim 9, wherein the miscible polar aprotic solvent is acetonitrile.

11. The method of claim 1, wherein the base is triethylamine.

12. The method of claim 1, wherein R₃ is C₁-C₄ alkyl or C₇-C₁₀ arylalkyl and the product of step A) is hydrolyzed prior to performing step B).

13. The method of claim 1, wherein the palladium is recovered with other solvents and reactants from the first Suzuki coupling reaction and the combination of palladium with the other solvents and reactants from the first Suzuki coupling reaction is added to the second Suzuki coupling reaction.

14. The method of claim 13, wherein some solvents are removed prior to addition to the second Suzuki coupling reaction.

15. The method of claim 13, wherein the palladium is isolated from the other solvents and reactants prior to addition to the second Suzuki coupling reaction.

16. The method of claim 1, wherein an acid is added to the first Suzuki coupling reaction product to form a mother liquor and a precipitate.

17. The method of claim 16, wherein the first Suzuki coupling reaction product contains one or more solvents and a portion of the one or more solvents are removed from the first Suzuki coupling reaction product prior to acid addition.

18. The method of claim 16, wherein the acid is sulfuric acid.

19. The method of claim 16, wherein the precipitate is washed to remove the palladium catalyst and form a precipitate wash.

20. The method of claim 19, wherein the precipitate is washed with a miscible polar aprotic solvent and water mixture.

21. The method of claim 19, wherein the mother liquor and the precipitate wash are combined and the combined mixture forms a phase-separated solution comprising an organic-rich layer and an aqueous layer.

22. The method of claim 21, wherein the combined mother liquor and precipitate wash is pH adjusted to aid the phase separation.

23. The method of claim 22, wherein the pH is adjusted using a base.

24. The method of claim 23, wherein the base is one or more of ammonium hydroxide, sodium hydroxide, or potassium hydroxide.

25. The method of claim 21, wherein the organic-rich layer contains palladium catalyst and the organic-rich layer is added to the second Suzuki coupling to provide the recovered palladium catalyst.

26. The method of claim 25, further comprising adding additional palladium catalyst to the second Suzuki coupling in addition to the recovered palladium catalyst.

27. The method of claim 21, wherein the neutralized phase separated solution is heated to greater than 30° C. to aid palladium accumulation in the organic-rich layer.

28. The method of claim 1, wherein the palladium remains soluble during palladium catalyst recovery.

29. The method of claim 28, wherein the palladium catalyst remains soluble between pH 0.1 and pH 12.

30. The method of claim 1, wherein the palladium is isolated in a solid phase when recovered and the solid phase is added to the second Suzuki coupling reaction.

31. The method of claim 21, wherein the organic-rich layer is separated and volatile organics are distilled from the organic-rich layer and the palladium catalyst is contained in distillation remains.

32. The method of claim 31, wherein the distillation remains are added to the second Suzuki coupling to provide recovered palladium.

33. A method for reclaiming palladium used in a Suzuki coupling reaction comprising:
A) performing a Suzuki coupling of a compound of Formula (II) 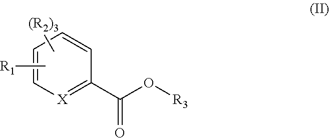
wherein
R₁ is halogen;
R₂ is H, halogen, —CN, —NO₂, formyl, C₁-C₆ alkyl, C₃-C₆ cycloalkyl, C₁-C₆ alkenyl, C₁-C₆ alkynyl, C₁-C₆ alkoxy, C₁-C₆ haloalkyl, C₁-C₆ haloalkenyl, C₁-C₆ haloalkynyl, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, aryloxy, heteroaryloxy, arylthio, heteroarylthio, NR₆R₇, or NHC(O)R₈;
R₃ is H, C₁-C₄ alkyl, or C₇-C₁₀ arylalkyl;
R₆, R₇ and R₈ are H or C₁-C₄ alkyl; and
X═CR₉ or N, wherein R₉ is H, halogen, NR₆R₇, or NHC(O)R₈,
and a compound of Formula (III) 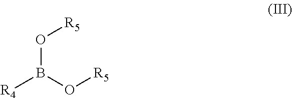
wherein
R₄ is a phenyl unsubstituted or substituted with 1-4 substituents independently selected from F, Cl, —CN, —NO₂, formyl, C₁-C₆ alkyl, C₃-C₆ cycloalkyl, C₁-C₆ alkenyl, C₁-C₆ alkynyl, C₁-C₆ alkoxy, C₁-C₆ haloalkyl, C₁-C₆ haloalkenyl, C₁-C₆ haloalkynyl, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, aryloxy, heteroaryloxy, arylthio, heteroarylthio, —NR₆R₇, or NHC(O)R₈ or a heteroaryl unsubstituted or substituted with from 1 to the maximum number of substituents independently selected from F, Cl, —CN, —NO₂, formyl, C₁-C₆ alkyl, C₃-C₆ cycloalkyl, C₁-C₆ alkenyl, C₁-C₆ alkynyl, C₁-C₆ alkoxy, C₁-C₆ haloalkyl, C₁-C₆ haloalkenyl, C₁-C₆ haloalkynyl, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, aryloxy, heteroaryloxy, arylthio, heteroarylthio, —NR₆R₇, or NHC(O)R₈;
R₅ is H, C₁-C₄ alkyl, or where the carbons on two R₅ are taken together to form a saturated ring as —O(C(R₁₀)₂)ₚO—, wherein p is 2 or 3; and
R₁₀ is H or C₁-C₄ alkyl,
using a palladium catalyst in the presence of a ligand and an amine base to form a Suzuki coupling reaction product;
B) isolating the palladium catalyst from the Suzuki coupling reaction product into a palladium catalyst isolate; and
C) substantially reclaiming the palladium catalyst from the palladium catalyst isolate.

34. The method of claim 33, wherein greater than 70% of the palladium catalyst used in the first Suzuki coupling reaction is recovered.

35. The method of claim 33, wherein the palladium catalyst isolate is formed by adding an acid to the Suzuki coupling reaction product to form a mother liquor and a precipitate.

36. The method of claim 35, wherein the acid is sulfuric acid.

37. The method of claim 35, wherein the precipitate is washed to remove the palladium catalyst and form a precipitate wash.

38. The method of claim 37, wherein the precipitate is washed with a solvent and water mixture.

39. The method of claim 38, wherein the solvent is a miscible polar aprotic solvent.

40. The method of claim 37, wherein the mother liquor and the precipitate wash are combined and the combined mixture forms a phase separated solution comprising an organic-rich layer and an aqueous layer.

41. The method of claim 40, wherein the combined mother liquor and precipitate wash is pH adjusted to aid the phase separation.

42. The method of claim 41, wherein the pH is adjusted using a base.

43. The method of claim 42, wherein the base is one or more of ammonium hydroxide, sodium hydroxide, or potassium hydroxide.

44. The method of claim 40, wherein the palladium catalyst is concentrated in the organic-rich layer.

45. The method of claim 44, wherein the palladium catalyst is extracted from the organic rich layer with ethyl acetate.

46. The method of claim 44, wherein the palladium catalyst is extracted from the organic-rich layer by adsorbing onto an organic substrate.

47. The method of claim 46, wherein the organic substrate is activated carbon.

48. The method of claim 33, wherein the palladium catalyst isolate is in a solid phase when isolated.
